(19) United States
(12) Patent Application Publication
(10) Pub. No.: US 2010/0024817 A1
(43) Pub. Date: Feb. 4, 2010

(54) INTRAORAL CPAP INTERFACE

(76) Inventor: David Madjar, Ramat Gan (IL)

Correspondence Address: DANIEL J SWIRSKY, 55 REUVEN ST., BEIT SHEMESH, 99544, IL

(21) Appl. No.: 12/442,402

(22) PCT Filed: Oct. 7, 2007
(86) PCT No.: PCT/IL07/01212

§ 371 Date: Mar. 22, 2009

(30) Foreign Application Priority Data
Oct. 4, 2006 (IL) 178441

(51) Int. Cl. A61M 16/00

(52) U.S. Cl. 128/204.18

(57) ABSTRACT

An intraoral continuous positive airway pressure (CPAP) interface that comprises a tube connected to a source of positive air pressure, and a shield connected to, or integrally formed with, the tube and adapted to be inserted within buccal sulci in such a way that facilitates oral cavity sealing. The shield has a central part formed with an aperture in communication with the tube, and right and left longitudinally extending projections adjoining, and of substantial bilateral symmetry with respect to the central part. Each of the projections has adjoining upper and lower regions and each of the regions has adjoining proximal and distal portions, Each of the projections is dimensioned such that a distal portion has a thickness substantially equal to, or greater than, a buccal sulcus potential space gap, and is configured, when inserted within a buccal sulcus, in such a way so as to adhere to the oral mucosa, to occupy the entire volume of buccal sulcus potential space, and to seal the oral cavity.

### FIELD OF THE INVENTION

The present invention relates to apparatus for alleviating sleep apnea. More particularly, the invention relates to an intraoral interface through which continuous positive air pressure, or any other suitable gas, is applied to a patient.

### BACKGROUND OF THE INVENTION

Obstructive sleep apnea (OSA) syndrome is a disorder characterized by repetitive episodes of upper airway obstruction that occur during sleep. Breathing disorders during sleep are considered as a public health problem, detracting from the quality of life due to reduced alertness and negatively influencing the cardiovascular system, resulting in increased morbidity and mortality. As positive pressure is applied in the upper airway by means of continuous positive air pressure (CPAP) devices, the airway remains unobstructed, thereby preventing apnea hypoxia and sleep disturbance.

Nasal CPAP treatments, by which a mask in communication with a source of air is fitted over the nose of a patient, have been shown to be successful in treating OSA and in reducing the frequency of occlusive and mixed patterns of OSA. However, compliance with nasal and oronasal CPAP treatment is problematic and declines considerably during time. Common problems with nasal CPAP mask include skin abrasions, mask pressure on facial structures, claustrophobia, air leaks, eye discomfort, nasal dryness and congestion. Full-face masks have the same limitations.

Recent studies indicate that oral positive air pressure is effective in treating OSA and produces a comparable upper airway flow as that of nasal positive air pressure. (Smith P L, Chest 2003)

The Oracle™ Mask manufactured by Fisher & Paykel Healthcare, USA, a CPAP device which applies positive pressure solely through the oral cavity has been introduced to overcome the known drawbacks of CPAP masks, but this oral mask is nevertheless bothersome in that it presses on the skin and lips. Also, the placement of this oral mask between the teeth and the intrusion thereof into the oral cavity disturb patient when sleeping.

WO 01/52928 discloses an intraoral apparatus for enhancing airway patency. The apparatus includes a member for providing mechanical positioning or retention of selected intraoral features, such as the tongue or soft palate, alone or in combination with the application of positive pressure. When this apparatus is inserted within the oral cavity, it is positioned lingually to the teeth, touching the teeth, tongue and soft palate. The apparatus is therefore cumbersome to the patient, and is particularly annoying when touching or pressing on the tongue, which is richly innervated by four sensory nerves, the glossopharyngeal nerve, the lingual nerve, and the facial and chorda tympany nerves. Another disadvantage of this apparatus is that it is liable to stimulate the hyperactive gag reflex which causes uncontrollable vomiting when touching the palate or tongue. Also, the occlusion and articulation patterns of the patient's dentition will invariably change as a result of the introduction of the apparatus lingually to the teeth. Over-eruption of the teeth may also occur if teeth are retained in a separated position for a prolonged time.

It is an object of the present invention to provide an intraoral device through which continuous positive airway pressure is delivered.

It is an additional object of the present invention to provide a CPAP device which does not press on the skin and lips externally to the oral cavity.

It is an additional object of the present invention to provide an intraoral CPAP device which applies positive pressure solely through the oral cavity,

It is an additional object of the present invention to provide an intraoral CPAP device which is not placed between the teeth and which does not protrude into the oral cavity lingual to the teeth.

It is an additional object of the present invention to provide a CPAP device which is comfortable and therefore encourages patient compliance.

It is yet an additional object of the present invention to provide a CPAP device which does not touch the tongue or soft palate.

It is yet an additional object of the present invention to provide a CPAP device which cannot stimulate the hyperactive gag reflex.

It is an additional object of the present invention to provide a CPAP device that will not change the occlusion and articulation patterns of a patient's dentition.

Other objects and advantages of the invention will become apparent as the description proceeds.

### SUMMARY OF THE INVENTION

The present invention provides an intraoral continuous positive airway pressure (CPAP) interface, comprising a tube in communication with a source of positive air pressure, and a shield connected to, or integrally formed with, said tube and adapted to be inserted within buccal sulci in such a way that facilitates oral cavity sealing.

As referred to herein, the following terms refer to the corresponding relative location of elements of the intraoral interface or of bodily portions:

“buccal”—toward the cheek or lip;
“lingual”—toward the tongue;
“proximal—toward the centerline of the shield;
“distal”—away from the centerline of the shield;
“longitudinal”—along the length of a shield, following the shape or contour of the teeth and substantially parallel to the centerline of the shield;
“intraoral”—disposed lingually to the lips”
“oral cavity”—the interspace lingual to the teeth and delimited by the palate and tongue;
“buccal sulcus”—a potential space between the attached gingiva and a lip which can be occupied when an interface shield is inserted therein; and
“potential space”—a gap in which material or a device is insertable to expand a flexible wall (lip) of the potential space, but which is contractable when the material or device is removed therefrom.

The shield is provided with a central part formed with an aperture in communication with the tube, and right and left longitudinally extending projections adjoining, and of substantial bilateral symmetry with respect to, said central part, each of said projections having adjoining upper and lower regions and each of said regions having adjoining proximal and distal portions.

Each of said projections is dimensioned such that a distal portion has a thickness substantially equal to, or greater than, a buccal sulcus potential space gap, and is configured, when inserted within a buccal sulcus, in such a way so as to adhere to the oral mucosa, to occupy substantially the entire volume of buccal sulcus potential space, and to seal the oral cavity. Due to the engagement of the distal portions with the oral mucosa, outflow of pressurized air from the oral cavity is prevented.

Each of the projections is preferably continuously adherable to the oral mucosa from the orbicularis oris muscle to the attached gingiva.

The shield has a longitudinal length equal to 10 to 16 teeth, and preferably 12 teeth.

A most distal location of a proximal portion and a transitional point between the orbicularis oris muscle and the buccinator muscle are approximately at a common height when the shield in inserted within the potential space of the buccal sulci.

In one aspect, a distal portion is considerably thicker than an adjoining proximal portion and than a corresponding distal portion of the buccal sulcus potential space to such a degree that upper and lower lip portions disposed bucally to the central part are urged to sealingly engage the tube.

The present invention is also directed to a method for applying an intraoral CPAP device to the mouth of a subject, comprising the steps of—

a) providing an intraoral CPAP device comprising a tube in communication with a source of positive air pressure, and a shield provided with a central part formed with an aperture in communication with said tube, and right and left longitudinally extending projections adjoining, and of substantial bilateral symmetry with respect to, said central part, each of said projections having adjoining upper and lower regions and each of said regions having adjoining proximal and distal portions, wherein each of said projections is dimensioned such that a distal portion has a thickness substantially equal to, or greater than, a buccal sulcus potential space gap; and
b) inserting said projections within a buccal sulcus in such a way so as to adhere to the oral mucosa, to occupy substantially the entire volume of buccal sulcus potential space, and to seal the oral cavity.

The method preferably further comprises the step of adapting the shield to the natural formation of the buccal sulcus potential space.

### BRIEF DESCRIPTION OF THE DRAWINGS

In the drawings:

FIG. 1 is a perspective view from the top of an intraoral interface, according to one embodiment of the invention;

FIG. 2 is a perspective view from the top of the intraoral interface of FIG. 1, to which an auxiliary tube is attached;

FIG. 3 is a picture of an interface shield, showing the lingual face thereof;

FIG. 4 a sagittal cross section of the oral cavity when an intraoral interface is engaged with the buccal sulci;

FIG. 5 is a sagittal cross section of the oral cavity, showing an unoccupied buccal sulci;

FIG. 6 is a vertical cross sectional of a shield engaged with the buccal sulci, illustrating distal and proximal portions thereof;

FIG. 7 is a vertical cross sectional of a shield engaged with the buccal sulci, illustrating the valve seal that is formed thereby;

FIGS. 8A and 8B illustrate an improperly shaped interface engaged with the buccal sulci, illustrating a reduction in the air pressure within the oral cavity;

FIGS. 9A-D illustrate an intraoral interface provided with an inflatable sleeve or an inflatable pouch; and

FIG. 10 illustrates a perspective view from the top of the lingual face of an intraoral interface provided with unidirectional valves for incoming and exhausted air.

### DETAILED DESCRIPTION OF PREFERRED EMBODIMENTS

The present invention is a novel intraoral continuous positive airway pressure (CPAP) interface, which is placed in the buccal sulcus, between the inner aspect of the cheeks and lips and the buccal aspect of the gums. While prior art CPAP devices are uncomfortable to the patient, particularly due to the pressure applied to various bodily parts, such as the lips, facial skin, and teeth, thereby reducing patient compliance, the interface of the present invention does not cause any significant discomfiture to the patient; it is configured in complementary fashion to the interspace of the buccal sulcus and therefore does not apply any significant pressure to the gums, lips or teeth. The interface is also configured such that, after being inserted within the buccal sulcus, it advantageously does not intrude between the teeth and between the upper and lower tooth arches, and does not contact portions of the tongue or of the oral cavity which are lingually disposed with respect to the teeth. Upon placement of the interface within the potential space of the buccal sulcus, the oral cavity becomes sealed by means of contact between perioral soft tissue and an interface shield, as will be described hereinafter, and pressurized CPAP gas, particularly air, which is delivered to the oral cavity via the interface is therefore prevented from escaping to the surroundings. Since the relatively high pressure of CPAP gas is retained in the oral cavity, the upper airway through which CPAP gas flows is therefore able to remain unobstructed and the manifestation of obstructive sleep apnea (OSA) is prevented or mitigated.

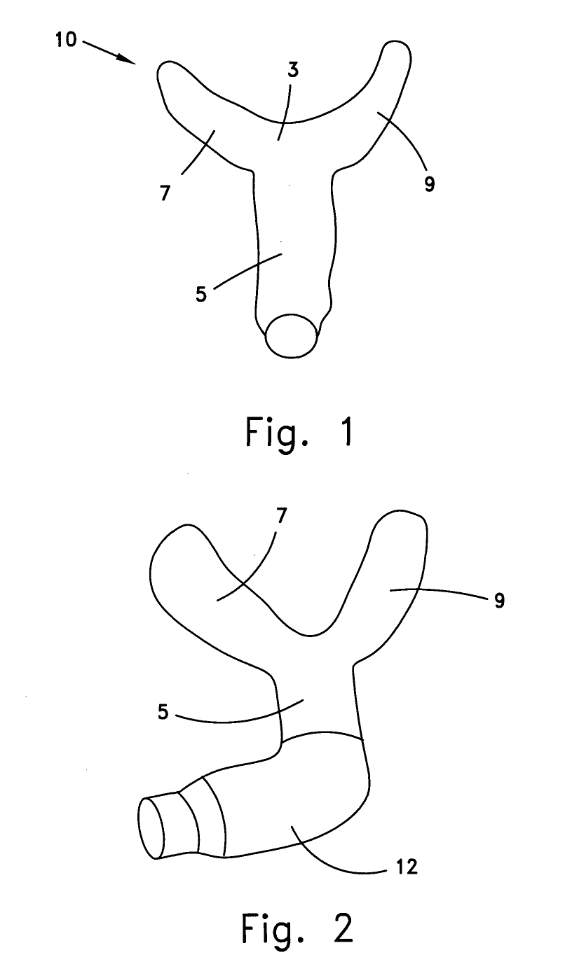

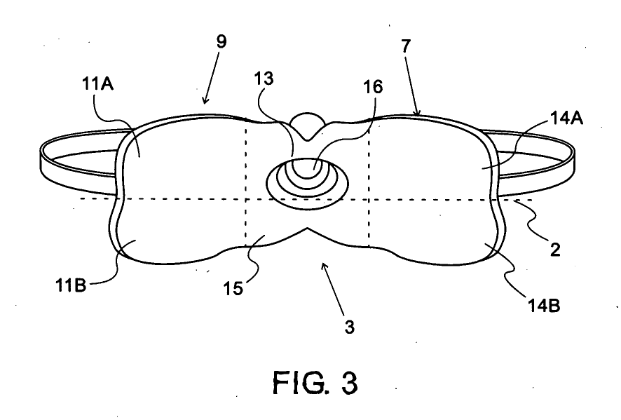

FIG. 1 illustrates a perspective view from the top of an intraoral interface, which is generally designated by numeral 10, according to one embodiment of the present invention. Interface 10 comprises tube 5 in communication with the source of positive pressure, and shield 3, which serves as a barrier between the oral cavity and atmospheric pressure air. Shield 3 has left and right arcuate projections 7 and 9 of bilateral symmetry with respect to tube 5. Projections 7 and 9 are configures to follow the shape and contour of the teeth of a patient. CPAP air is delivered through tube 5 and is discharged therefrom via aperture 16 into the oral cavity. The diameter of tube 5 is sized to ensure to a suitable flow rate of CPAP to retain the airway unobstructed. As shown in FIG. 2, tube 5 is engageable with auxiliary tube 12 of a different orientation, e.g. a difference in orientation of 90 degrees, than interface tube 5. Auxiliary tube 12 functions as an adaptor to a hose through which CPAP air flows from a CPAP generator, e.g. a compressor or air pump, to interface 10. Tube 5 is rotatable with respect to auxiliary tube 12, or vice versa, to advantageously afford the patient much mobility when asleep without concern that the supply of CPAP air will be disrupted.

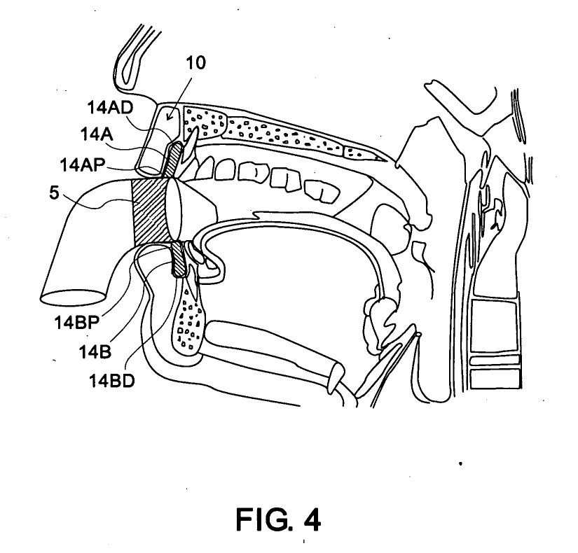

FIG. 3 is a picture of the lingual side of shield 3. As shown, shield 3 is provided with a central part 15 which is formed with an aperture 16 in communication with tube 5. Peripheral portion 13 surrounds aperture 16. When shield 3 is inserted within the buccal sulci, central part 15 is generally positioned within the buccal sulci between the right and left canine teeth. Projections 7 and 9 adjoin central part 15 and are arranged such that projection 7 has adjoining upper and lower regions 14A and 14B, and projection 9 has adjoining upper and lower regions 11A and 11B. Centerline 2 of shield 3 denotes the border between upper and lower regions 14A and 14B. Central part 15 may be formed with a greater hardness than the other portions of shield, to allow tube 5 to be engaged with auxiliary tubing 12 or with any other commercially available tubing or air flow equipment.

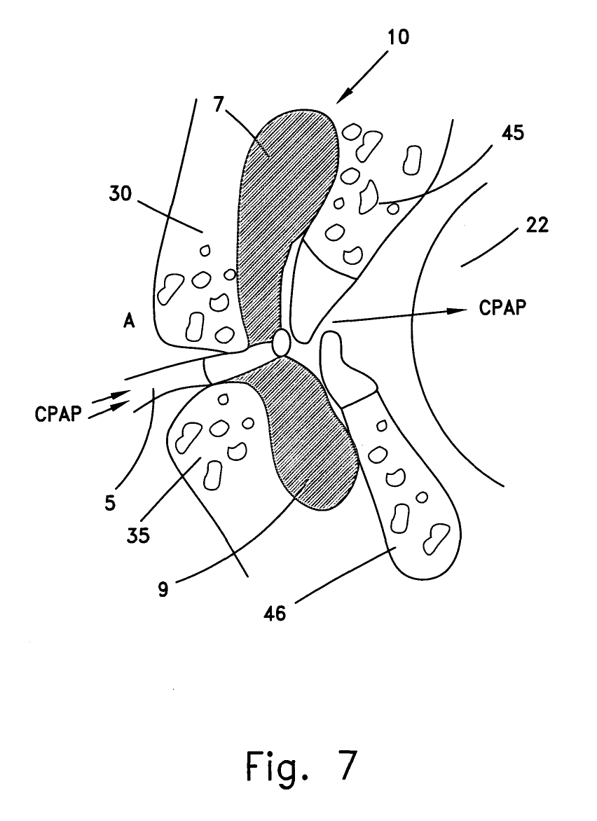

FIG. 6 illustrates a schematic vertical cross sectional view of the interface after being inserted in the upper and lower buccal sulci. Each region of the shield has adjoining proximal and distal portions. As shown, upper region 14A has proximal portion 14AP and distal portion 14AD, and lower region 14B has proximal portion 14BP and distal portion 14BD. A distal portion has a considerably larger thickness than the adjoining corresponding proximal portion. The height of regions 14A and 14B, i.e. the vertical dimension from the corresponding proximal portion to the corresponding distal portion, is greater than the diameter of tube 5. Distal portions 14AD and 14BD may be more lingually disposed than proximal portions 14AP and 14BP, respectively.

The dimensions of interface 10 are preferably customized, so that the shield may be snugly fit within the buccal sulcus of a given patient. Suitable interface materials that are easily adaptable to the individual size of a patient include soft biocompatible materials, thermoplastic materials, soft moldable silicon, therapy putty of a relatively soft grade, and rubberized materials, or a combination thereof.

The shield may be made of one material. The shield may also be made from more than one section. For example, the shield may be formed with a first inner section made of a relatively hard material, e.g. silicon having a high hardness, and a second softer section surrounding the first section, e.g. low-hardness silicon in contact with perioral soft tissue, for increasing the shear resistance of the shield. The shield may also be made from three sections. The first section coincides with central part 15 (FIG. 3) and is relatively hard. The second section is softer than, and surrounds, the first section. The third section surrounds the second section, e.g. constituting the periphery of the shield, and is made of material which is even softer than the second section. To strengthen the third section which is engaged with the perioral tissue, the third section may be reinforced with a skeleton member, e.g. in the form of a string. The separation of the shield material into the second and third sections does not necessarily coincide with the separation of the shield configuration into proximal and distal portions.

Alternatively, the shield may be made of two or more materials that harden at body temperature. Each material may be confined in a different chamber within the shield and separated by a breakable partition. After the patient inserts the shield into the buccal sulci, the partition is broken by a motion that will cause the materials to mix and cure, so that the shield projections will occupy substantially the entire potential space of the buccal sulci.

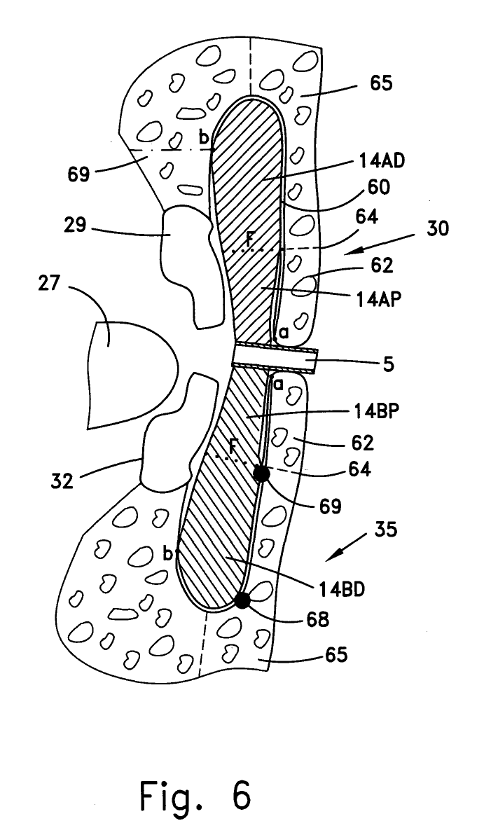

In order to explain the utility of the present invention, reference is first made to FIG. 5, which illustrates a sagittal cross section of the oral cavity. As shown, oral cavity 22 is delimited by hard palate 25 from above, tongue 27 from below, and by teeth 29 and 32 at the buccal borders thereof. Upper buccal sulcus 31 is between upper teeth 31 and upper lip 30. Lower buccal sulcus 34 is between lower teeth 32 and lower lip 35. Upper airway 39, which remains unobstructed by use of the intraoral interface of the present invention, is in communication with oral cavity 22. However when the intraoral interface of the present invention is not in use, a person suffering from the OSA syndrome often suffers from sleep fragmentation, daytime sleepiness, and cardiac dysrythmias due to the oxygen deprivation resulting from complete or partial airway obstruction.

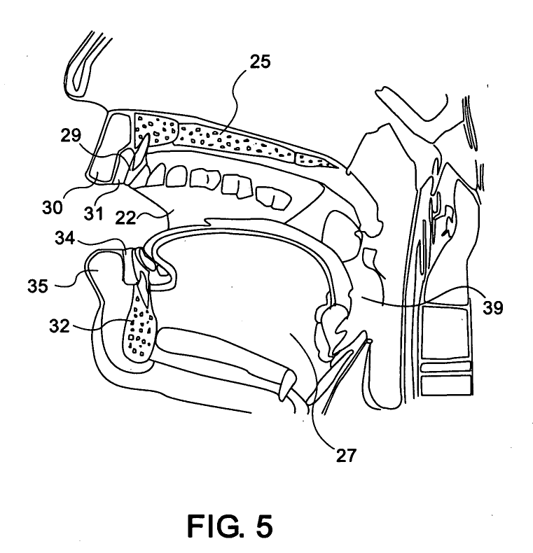

Referring to FIGS. 4 and 5, placement of intraoral interface 10 in the upper and lower buccal sulci 31 and 34 ensures that positive air pressure is admitted to oral cavity 22 via tube 5, in order to retain the airway unobstructed and to prevent the occurrence of OSA. Upper and lower shield regions 14A and 14B have the same general shape as upper and lower buccal sulci 31 and 34, respectively, and may be self-adaptable to the dimensions and general shape of the buccal sulci. For example, the distal portions of each shield region are more lingually disposed than proximal portions thereof when the corresponding distal portions of the buccal sulcus are more lingually disposed than proximal portions thereof. Proximal portions 14AP and 14BP are therefore able to snugly fit in the proximal portions of upper and lower buccal sulci 31 and 34, respectively. Since distal portions 14AD and 14BD are considerably thicker than proximal portions 14AP and 14BP, respectively, and upper and lower buccal sulci 31 and 34, respectively, each distal shield portion presses on the corresponding distal wall portion of the buccal sulcus adjoining the corresponding lip. As a distal lip portion of upper and lower lips 30 and 35, which is composed of the flexible buccinator muscle, is buccally pressed by the corresponding distal shield region portion, a proximal lip portion being composed of relative rigid orbicularis oris muscle which is not pressed by a shield region is urged more proximally. By virtue of the oral musculature commonly referred to by those skilled in the art of oral rehabilitation as the “neutral zone philosophy application” in which moldable materials needed for the stabilization of dentures are placed and shaped in the buccal sulcus, perioral soft tissue such as the oral mucosa is retained in abutting and pressing relation with a corresponding shield distal portion. The lips adjacent to the central part of the interface are also urged to be in sealing engagement with tube 5 as shown.

In contrast to prior art intraoral CPAP devices which are adapted to touch or press on the tongue being innervated by four sensory nerves and thereby causing considerable discomfiture to the patient, the bodily portion to which a force is applied and by which patient compliance is made possible is the oral mucosa, the lingual mucous membrane located within the epithelium or lining of the lips. The relative location of the oral mucosa is schematically illustrated in FIG. 6.

As shown in FIG. 6, oral mucosa 60 lines the lingual wall of lips 30 and 35, extending from point a, which corresponds to the most distal portion of the buccal sulcus potential space, to point b, which corresponds to the termination of attached gingiva 69. Lips 30 and 35 are composed of the relatively rigid, circular ring orbicularis oris muscle 62 at a proximal portion thereof and of the relatively flexible buccinator muscle 65 at a distal portion thereof. The oral mucosa is innervated by the maxillary and mandibular nerves, the nerve ending density of which is significantly less than the total nerve ending density of the four sensory nerves of the tongue. As a result of the natural flexibility of the lips and the presence of low-sensitivity tactile sensors adjoining the buccal sulci, it will be appreciated that the use of interface 10 advantageously causes essentially no discomfiture to the patient as the perioral soft tissue is urged against projections 7 and 9 and lips 30 and 35 are urged against central part 15 (FIG. 3) of the shield, thereby increasing patient compliance.

The shield may be configured such that the most distal location F of proximal portions 14AP and 14BP, which is represented by a dotted line, may be at a substantially common height as the transitional point between orbicularis oris muscle 62 and buccinator muscle 65, which is schematically illustrated by dashed line 64, when the shield in inserted within the potential space of the buccal sulci. Orbicularis oris muscle 62 may then press on proximal portions 14AP and 14BP. The shield may therefore be stabilized thereby even though proximal portions 14AP and 14BP are configured such that they do not adhere to oral mucosa 60. A shield made of silicon may advantageously adhere to attached gingiva 69.

In another embodiment, the shield comprises a springy element 68 or 69 protruding from the buccal surface of distal portions 14AD and 14BD, although the elements are shown only with respect to portion 14BD. Element 68 is shown to be attached to the most distal section of the buccal face of the shield. Element 69 is shown to be attached to the most proximal section of distal portions 14AD and 14BD. A proximally extending strip is connected to each of the springy elements. The strip connected to springy element 68 covers the entire buccal surface of the corresponding extension. The strip connected to springy element 69 covers the entire buccal surface of the corresponding proximal portion. The strip additionally induces contact between the shield and the perioral tissue by following the movement of the lips to enhance the seal.

In another embodiment, the shield comprises a proximally extending strip 68 or 69 connected to the buccal surface of distal portions 14AD and 14BD, although the strips are shown only with respect to portion 14BD. Strip 68 is shown to be attached to the most distal section of the buccal face of the shield, and covers the entire buccal surface of the corresponding extension. Strip 69 is shown to be attached to the most proximal section of distal portions 14AD and 14BD, and covers the entire buccal surface of the corresponding proximal portion. The strip additionally induces contact between the shield and the perioral tissue by following the movement of the lips to enhance the seal.

When the perioral soft tissue in an abutting and pressing relationship with the distal and proximal shield portions as shown, the soft tissue constitute a seal to prevent the outflow of atmospheric air from the oral cavity and to maintain the necessary positive air pressure needed to prevent the manifestation of OSA. The soft tissue seal utilizes the phenomenon commonly referred to by those skilled in the art of oral rehabilitation as the “valve seal”. Dentures are generally retained in a toothless mouth by means of the valve seal whereby the potential space of the buccal sulcus, when filled with moldable material, contact, and are displaced by, the borders of the denture. This intimate contact prevents air from infiltrating under the denture, thereby producing a seal.

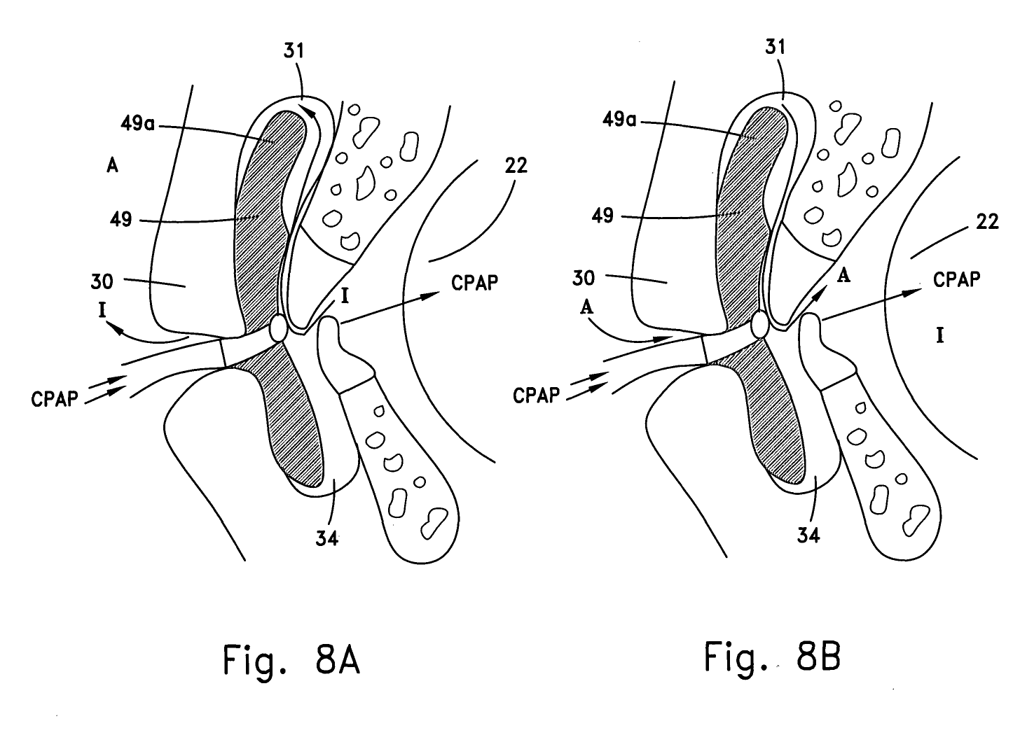

FIG. 7 illustrates the generation of a valve seal when an optimally shaped interface 10 is placed in, and completely fills, the buccal sulci. Due to the presence of oral mucosa and a thin film of saliva on the lingual surface of lips 30 and 35, the interfacial surface tension between projections 7 and 9 and the oral mucosa resists separation of interface 10 from the buccal sulci. A hermetic seal that secludes oral cavity 22 from outside air A is consequently generated, thereby allowing the air pressure within oral cavity 22 lingual to the teeth to increase. CPAP air can be therefore introduced to oral cavity 22 via tube 5; however pressurized air within oral cavity 22 is prevented from escaping via a buccal sulcus and atmospheric pressure air A is prevented from infiltrating to oral cavity 22 through a buccal sulcus by utilizing the phenomenon of the valve seal.

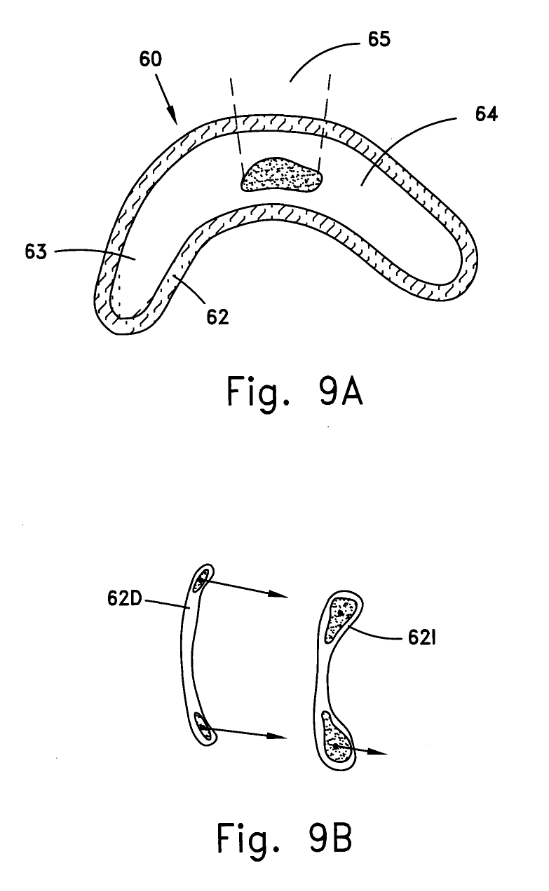

FIGS. 8A and 8B schematically illustrate the reduction of air pressure within oral cavity 22 when an improperly shaped interface is employed. As shown, the distal portion 49a of the shield has a significantly smaller thickness than the gap of buccal sulci 31 and 34. Consequently, an unfilled space not occupied by distal portion 49a results. Due to the lack of interfacial surface tension between distal portion 49a and the oral mucosa, distal portion 49a becomes separated from the lingual surface of lip 30. In FIG. 8A, pressurized air I resulting from CPAP which is internal to oral cavity 22 is shown to escape from oral cavity 22 to atmospheric pressure air A via buccal sulci 31 and 34. In FIG. 8B, atmospheric air A is shown to infiltrate through the small clearance that is formed between interface 49 and lip 30. Atmospheric air A subsequently enters oral cavity 22 and mixes with CPAP air that is delivered thereto, thereby reducing the pressure of positive air within the airway necessary for the prevention of OSA. It will be appreciated that the outflow of pressurized air I is more prevalent than the infiltration of atmospheric air A.

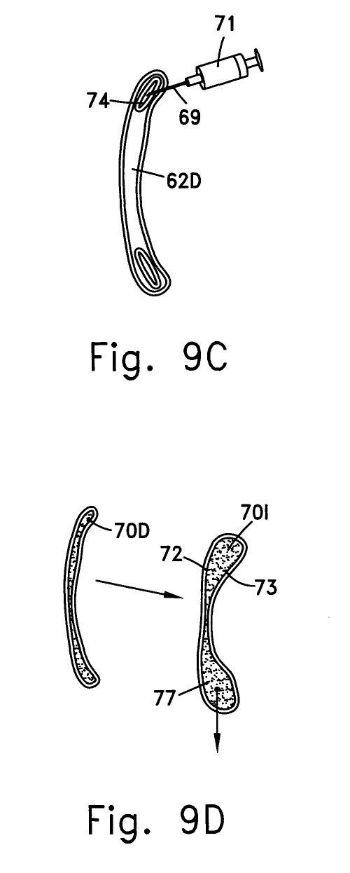

FIGS. 9A-D illustrate another embodiment of the invention wherein the projections are inflatable sleeves.

Interface 60, which comprises projections 63 and 64 embodied by an inflated sleeve 62 formed with a sealed aperture in communication with tube 65, is schematically illustrated in FIG. 9A. Sleeve 62 is inflated until it fills the buccal sulcus so that a valve seal will be generated. FIG. 9B illustrates the difference between a deflated sleeve 62D and an inflated sleeve 621. The distal portions of inflated sleeve 62I are shown to be significantly wider than the central part thereof. The sleeve can be easily inflated as shown in FIG. 9C by any suitable medium 69, such as the illustrated liquid silicon, or by water, air, or by a combination of materials. The medium 69 can also be introduced into sleeve 62D via an accessible port 74 by the patient by means of a hand held injector 71. The shield may therefore be self adaptable by injecting medium 69 until the patient senses that the distal portions engage the oral mucosa.

In FIG. 9D, a deflated sleeve 70D and inflated sleeve 70I made of two layers 72 and 73 of soft moldable silicon, or of any other biocompatible soft material, and a sealed pouch 77 therebetween are illustrated. Pouch 77 can be filled with liquid or gas as described hereinabove. The liquid pressure or the compressed gas pressure on the soft silicon will allow sleeve 701 to be adapted to the shape of the buccal sulcus, to help generate a valve seal. An additional way for inflating the adaptable sleeve may be by using part of the air delivered by the CPAP machine to inflate the adaptable sleeve through a special duct or ducts. Control of the air pressure in the sleeve may be achieved for example by evacuating the air from the sleeve into the oral cavity through smaller diameter ducts as compared to the incoming air duct.

The sleeves and/or pouch may contain two or more materials that harden at body temperature. Each material may be confined in a different chamber within each sleeve and/or pouch and separated by a breakable partition. After the patient inserts the shield into the buccal sulci, the partition is broken by a motion that will cause the materials to mix and cure, so that the shield projections will occupy substantially the entire potential space of the buccal sulci.

An intraoral interface may be sold in several sizes, such as one for adult sizes, children sizes, and infant sizes. An interface may also be sold as a kit which allows adaptation to the natural buccal sulcum formation of a patient. An adaptable shield may be further customized by being sold according to the size of adults, children, or adults.

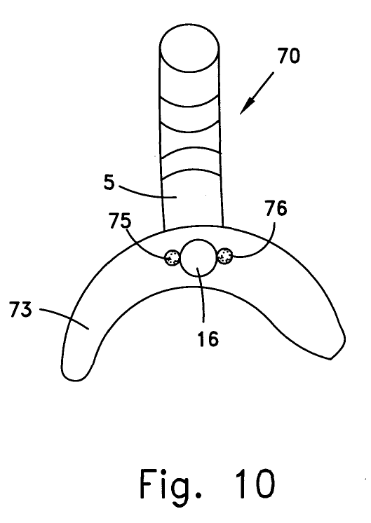

As schematically illustrated in FIG. 10, the air supply from the positive pressure generator may be delivered to interface 70 and to the oral cavity via narrow tubing used in prior art nasal CPAP interfaces, tube 5, aperture 16, and unidirectional safety valve 75 operationally connected to the central part of the shield, to ensure that air will continue to be delivered if the positive pressure generator will malfunction. Interface 70 may also comprise unidirectional exhaust valve 76 that can be adjusted to control the flow rate of exhaust gas to the atmosphere, so that end expiratory positive air pressure may be regulated. Exhaust valve 76 can also be operatively connected to tube 5 or auxiliary tube 12 (FIG. 2). It will be appreciated that exhaust gas may also be vented by means of a Whisper swivel, which is well known to those skilled in the art, between tube 5 and auxiliary tube 12.

It will be appreciated that the interface of the present invention can be used in conjunction with other respiratory fields, such as the delivery of positive pressure gas, e.g. oxygen, to a patient.

While some embodiments of the invention have been described by way of illustration, it will be apparent that the invention can be carried out with many modifications, variations and adaptations, and with the use of numerous equivalents or alternative solutions that are within the scope of persons skilled in the art, without departing from the spirit of the invention or exceeding the scope of the claims.

1-13. (canceled)

14. An intraoral continuous positive airway pressure (CPAP) interface, comprising a tube in communication with a source of positive air pressure, and a shield connected to, or integrally formed with, said tube and adapted to be inserted within buccal sulci in such a way that facilitates oral cavity sealing.

15. The interface according to claim 14, wherein the shield is provided with a central part formed with an aperture in communication with the tube, and right and left longitudinally extending projections adjoining, and of substantial bilateral symmetry with respect to, said central part, each of said projections having adjoining upper and lower regions and each of said regions having adjoining proximal and distal portions, wherein each of said projections is dimensioned such that a distal portion has a thickness substantially equal to, or greater than, a buccal sulcus potential space gap, and is configured, when inserted within a buccal sulcus, in such a way so as to adhere to the oral mucosa, to occupy substantially the entire volume of buccal sulcus potential space, and to seal the oral cavity.

16. The interface according to claim 15, wherein each of the projections is continuously adherable to the oral mucosa from the orbicularis oris muscle to the attached gingiva.

17. The interface according to claim 14, wherein the shield has a longitudinal length equal to 10 to 16 teeth.

18. The interface according to claim 17, wherein the shield has a longitudinal length equal to approximately 12 teeth.

19. The interface according to claim 15, wherein a most distal location of a proximal portion and a transitional point between the orbicularis oris muscle and the buccinator muscle are approximately at a common height when the shield in inserted within the potential space of the buccal sulci.

20. The interface according to claim 15, wherein a distal portion is considerably thicker than an adjoining proximal portion and than a corresponding distal portion of the buccal sulcus potential space to such a degree that upper and lower lip portions disposed bucally to the central part are urged to sealingly engage the tube.

21. A method for applying an intraoral CPAP device to the mouth of a subject, comprising the steps of:
a) providing an intraoral CPAP device comprising a tube in communication with a source of positive air pressure, and a shield provided with a central part formed with an aperture in communication with said tube, and right and left longitudinally extending projections adjoining, and of substantial bilateral symmetry with respect to, said central part, each of said projections having adjoining upper and lower regions and each of said regions having adjoining proximal and distal portions, wherein each of said projections is dimensioned such that a distal portion has a thickness substantially equal to, or greater than, a buccal sulcus potential space gap; and
b) inserting said projections within a buccal sulcus in such a way so as to adhere to the oral mucosa, to occupy substantially the entire volume of buccal sulcus potential space, and to seal the oral cavity.

22. The method according to claim 21, further comprising the step of adapting the shield to the natural formation of the buccal sulcus potential space.

23. The interface according to claim 14, further including one or more ducts for inflating an adaptable sleeve using part of the air delivered by the CPAP machine.

24. The interface according to claim 14, in which the air pressure in the sleeve is controlled by evacuating the air from the sleeve into the oral cavity through ducts having diameter which is smaller than the diameter of an incoming air duct.
